(19) United States
(12) Patent Application Publication
(10) Pub. No.: US 2008/0300221 A1
(43) Pub. Date: Dec. 4, 2008

(54) Contact Lens Care Composition

(76) Inventor: Andreas Obwaller, Vienna (AT)

Correspondence Address: CHALKER FLORES, LLP, 2711 LBJ FRWY, Suite 1036, DALLAS, TX, 75234, US

(21) Appl. No.: 11/817,011

(22) PCT Filed: Feb. 20, 2006
(86) PCT No.: PCT/AT2006/000063

§ 371 Date: Aug. 23, 2007

(30) Foreign Application Priority Data
Feb. 23, 2005 (AT) A 305/2005

(51) Int. Cl. A61L 12/14; A01P 1/00

(52) U.S. Cl. 514/77

(57) ABSTRACT

The present invention relates to ophthalmic compositions such as contact lens care solutions, containing miltefosine. The present invention further relates to ophthalmic compositions or contact lens care systems containing miltefosine in combination with additional other active microbiocidal substances or proteases. Furthermore, the invention relates to the use of the compositions for the removal and eradication of protozoan parasites such as Acanthamoeba from contact lenses.

The present invention relates to compositions with enhanced antimicrobial efficacy against Acanthamoeba.

Acanthamoeba keratitis is a corneal disease predominantly associated with contact lens wear. The occurrence of acanthamoeba keratitis has been rising since 1990 in correlation to the enhanced number of contact lens wearers. Approximately 3000 cases of acanthamoeba keratitis have been reported around the world. The disease usually progresses slowly, however, in most cases it ends up in a fulminant infection, very often leading to severe loss of vision and sometimes even to the enucleation of the afflicted eye. Meanwhile, Acanthamoeba are, besides the pseudomonads, the most common causative agents of contact lens-associated keratitis. It is estimated that the annual incidence of acanthamoeba keratitis in the USA is 1:250,000 inhabitants. In Europe most cases have been documented in the UK (around 400).

Contaminated contact lenses, lens cases or contact lens care systems usually are the first step in an acanthamoeba keratitis pathogenesis. Evidence of the ease with which human infection can potentially occur is reflected by the isolation of Acanthamoeba from water drawn from bathroom taps and from dust around a washbasin. Since Acanthamoeba species are ubiquitous in the environment, lens care systems could possibly even become contaminated with dust from the air. The presence of bacteria in contact lens cases and solutions originating from tap water or elsewhere predisposes to ocular Acanthamoeba infection. The amoeba feed on bacteria and multiply, with the result that large potential amoebic inocula may be present.

Several studies have shown that the effect of disinfectants against Acanthamoeba is insufficient and varies between different strains. Development of resistances and amoebostatic rather than amoebicidic effects are counter indicators of current drugs. Thus, a need for compositions for inhibiting growth of protozoans remains, such as Acanthamoeba in or on eye care products such as contact lenses, contact lens solutions and contact lens cases, in order to reduce the incidence of acanthamoebic keratitis and other ophthalmic pathologies due to the presence of protozoans. Current drugs administered in cases of acanthamoeba keratitis include propamidine isethionate, chlorhexidine gluconate and polyhexamethylene biguanide (Schuster et al., Cur. Treatm. Opt. Inf. Dis. 5:273-282, 2003).

General disinfecting contact lens care compositions are known in the state of the art and are disclosed e.g. in the WO 04/058930 A1. Such general disinfectants have been tested (see examples) and proven to be ineffective against Acanthamoeba contaminations on contact lenses.

The WO 2004/030710 A1 describes ophthalmic compositions for use as eye drop or contact lens cleaning solutions which comprise antimicrobial polycationic materials. Such polycationic materials are for example cationic cellulose derivatives with polyvalent cation chelating moieties, for example quaternary amino groups, which effectively inhibit protozoan cell function, particularly cell growth.

The GB 2 329 126 A describes disinfectant solutions containing polyhexamethylene biguanides.

Silvany et al. (Opthalmology 97(3) 286-90 (abstract), 1990) analysed contact lens disinfectants against Acanthamoeba. The most effective compound under investigation was chlorhexidine.

The GB 2 333 609 A describes a solution of sodium salicylate which inhibits the attachment of Acanthamoeba to contact lenses.

Furthermore, potential ocular drugs or preservatives have to fulfill certain criteria, e.g. deleterious effects on the tear film stability have to be avoided. The tear film is a three-layered fluid of 7 μm thickness composed of lipid, aqueous and mucin phases (Holly et al., Exp. Eye Res. 15:515-525, 1973). Any discontinuity and instability of this tear film over the cornea may lead to ocular non-wetting tears disorders called dry eye. Tear disorders are assessed by different clinical tests including break-up time test, the Schirmer test, fluorescein and rose bengal staining and biomicroscopy (Stulting et al. in Leibowitz, Corneal Disorders, Clinical Diagnosis and Management, W. B. Saunders, Philadelphia, Pa., pp. 445-468, 1984). Especially surface-active agents could solubilize the lipid layer of the tear film, which would lead to tear film rupture (Furrer et al., Eu. J. Pharm. Bioph., 53:262-280, 2002).

In a recent study it has been shown that miltefosine (1-hexadecylphosphorylcholine, also known as hexadecylphosphocholine, also referred to as HePC) shows high amoebicidal activity against trophozoites and cysts of three strains of Acanthamoeba of different pathogenicity. The treatment with miltefosine resulted in vacuolization, rounding up of cells, blebbing, and finally complete lysis of the cells after less than 30 minutes. (Walochnik et al., Antimicrob. Agents Chemother. 46: 695-701, 2002). Commercially available multi-purpose solutions, however, showed only (minor) reactivity against trophozoites and not against cysts (Hiti et al. Br J Opthalmol. 86: 144-6, 2002).

Miltefosine is a detergent-like drug with both ionic and lipophilic characteristics. It is an ester of phosphorylcholine and hexadecanol and therefore resembles a membrane-active alkylphospholipid. It is an inhibitor of the CTP phosphocholine cytidylyl transferase and has antimetastatic properties. Additionally, it is a very effective drug against visceral infections of Leishmania. Leishmania have high levels of ether-lipids and miltefosine is believed to act on key enzymes involved in the metabolism of ether lipids (More et al., J. Postgrad. Med. 49:101-103, 2003). Leishmaniasis is a very virulent tropical disease and transmitted by sand flies. Symptoms of visceral leishmaniasis include fever, spleen and liver enlargement, blood deficiencies, bleeding of mucous membranes and severe weight loss. Cutaneous leishmaniasis, although not lethal, is a severely disfiguring condition. Miltefosine (Impavido®) is licensed in most states for the oral treatment of leishmaniasis.

The technical problem underlying the present invention is to provide means and methods which allow the efficient prophylaxis against protozoan eye infections, especially in the field of contact lens care. A special goal of the present invention is to provide means to prevent Acanthamoeba infections transmitted through contact lens wear.

It is based on the use of hexadecylphosphocholine to eliminate the Acanthamoeba exactly at their major route into the human eye before they can do any damage. It contributes to saving health care cost by exercising prophylaxix rather than treatment.

Although the efficacy of miltefosine against Acanthamoeba is known in the prior art, practical uses of this compound have been restricted for the oral treatment of leishmaniasis so far. Factors like lipophilicity and surface-active properties of miltefosine appear to have been counter-indicators for the use in a composition as presented herein.

Surprisingly, the composition of the present invention did not exhibit negative effects, which allows the use in an ophthalmic composition or lens care system with amazing efficiency against protozoan germs such as Acanthamoeba.

The present invention provides contact lens care compositions containing miltefosine as antimicrobial agent in an amount effective for disinfecting contact lenses. Miltefosine therein is the primary amoebicidal substance, preferably used for eradication of Acanthamoeba and protozoan organisms from contact lenses. Preferably, the Acanthamoeba strains belong to A. castellani, A. hachetti and A. polyphaga.

Compositions for use in connection with the eye and with contact lenses which are subsequently placed in the eye are desirable such as to avoid discomfort and damage to the eye. Certain antibacterial compositions currently in use employ bactericides which, if used in connection with hydrophilic soft contact lenses, may be absorbed and concentrated in the lens. Such a lens, if placed in the eye, can release a concentrated solution leading to irritation of the eye. Therefore, ophthalmic compositions consist of substances, which can mediate effects of or alter the solubility of the specific antimicrobial substances employed. Usual additional substances in ophthalmic compositions are buffers, surfactants, viscosity increasing components, tonicity components and chelating agents.

The present invention can be used with all contact lenses such as conventional hard, soft, rigid and soft gas permeable lenses, however use of the present invention in soft lens case systems being especially advantageous. The compositions of the present invention may be aqueous or non-aqueous. Aqueous solutions are preferred. The composition according to the invention can also be pressed in a tablet form. Before use, the tablets can be dissolved in water or any other solution used for contact lens care systems. If the composition is provided in solid form, all concentrations given in the specification and the claims of the present invention refer to concentrations the compounds of the composition have after appropriate salvation.

The concentration of miltefosine in a preferred embodiments of the composition of the present invention is between 5 and 500 μM, further preferred between 10 and 300 μM, even further preferred between 20 and 200 μM.

A preferred implementation of the composition according to the present invention further comprises buffer components (phosphate salts or other buffering salts, such as TRIS) in an amount effective in maintaining the pH of the solution within a physiologically acceptable range, preferably with a pH between 6 and 8.

The composition may also comprise a surfactant in an amount effective in cleaning a contact lens. Examples of surfactants are poloxamines, poloxamers, alkyl ethoxylates, alkyl phenyl ethoxylates or other non-ionic, anionic and dipolar ionic surfactants known in the art.

A further implementation of the composition of the present invention additionally comprises a viscosity increasing-component, preferably selected from the group of cellulose derivatives, polyols, polyethylene oxide (PEO) containing polymers, polyvinyl alcohol and polyvinyl pyrrolidone. Cellulose derivatives are for example cationic cellulose polymers, hydroxypropyl methylcellulose, hydroxyethylcellulose and methylcellulose. An example of a polyol is polyethylene glycol.

The composition of the present invention furthermore preferably comprises a tonicity component, preferably salts, such as sodium chloride, potassium chloride and calcium chloride, or non-ionic tonicity agents, preferably propylene glycol or glycerol. Practical tonicities are in a preferred range of 200-600 mOsm, in an even more preferred range between 250-450 mOsm.

A further implementation of the composition of the present invention additionally comprises a chelating component, preferably ethylenediaminetetraacetic acid, alkali metal salts of ethylenediaminetetraacetic acid or mixtures thereof.

A further implementation of the composition of the present invention additionally comprises a protease. Proteases (enzymatic cleaners) are especially useful for contact lens wearers susceptible to high levels of protein deposits.

A further implementation of the composition of the present invention additionally comprises further disinfectants. An additional disinfectant, beside miltefosine, can be employed as functional preservative, but it may also function to potentate, complement or broaden the spectrum of the microbiocidal activity of another germicide. Suitable antimicrobial components are those generally employed in ophthalmic applications and include, but are not limited to, quaternary ammonium salts used in ophthalmic applications such as poly[dimethylimino-2-butene-1,4-diyl]chloride, alpha-[4-tris(2-hydroxyethyl)ammonium]-dichloride, benzalkonium halides, and biguanides, such as salts of alexidine, alexidine-free base, salts of chlorhexidine, hexamethylene biguanides and their polymers, and salts thereof, antimicrobial polypeptides, chlorine dioxide precursors, and the like and mixtures thereof. Generally, the hexamethylene biguanide polymers (PHMB), also referred to as polyaminopropyl biguanide (PAPB), is preferred.

As mentioned above, the composition of the present invention can be provided in solid state. Therefore, the present invention provides a miltefosine containing composition, for the cleaning of contact lenses, pressed in a tablet form.

The present invention encompasses the use of a composition containing miltefosine for cleaning, chemical disinfection, storing or rinsing contact lenses.

A further use of a composition according to the present invention relates to the elimination of protozoan organisms from a contact lens.

Preferably the composition of the present invention is used for the elimination of Acanthamoeba from a contact lens.

The present invention furthermore comprises the use of the present contact lens care composition, wherein the contact lens is selected from hard, soft, rigid or soft gas permeable lenses, preferably soft lenses.

The composition of the present invention can be used as storing solution for contact lenses in order to prevent contaminations of Acanthamoeba and other protozoan organisms. Therefore, the present invention encompasses the method for the purification of contact lenses, wherein the contact lens is stored in the composition according to the present invention for at least 1 minute.

A preferred implementation of the invention is a “multi-purpose solution” containing the composition of the present invention. The term “multi-purpose solution” means that the solution may be daily or periodically used for cleaning, chemical disinfection, storing, and rinsing contact lenses.

The present invention also comprises a “multi-purpose solution system” or “multi-purpose solution package” or a “kit”, which comprises the composition of the present invention together with rinsing solutions and/or drop delivery tools, preferably pipettes. Rinsing solutions can be appropriate sterilized buffer solutions or artificial tears. Such a kit may contain the composition of the present invention in packages (tablets or solution packages) indicated for the daily usage.

As an illustration of the present invention, several examples are provided below. These examples serve only to further illustrate aspects of the invention and should not be construed as limiting the invention.

The activity of various multi-purpose contact lens solutions (for cleaning, rinsing, storing and disinfecting a contact lens while the lens is not worn) was tested against cysts of A. castellani.

### EXAMPLES

### Example 1

### Miltefosine and Contact Lens Solutions

Contact lens disinfection solutions tested (Table 1) were purchased from local retail stores. In case of the H₂O₂ based systems, neutralization of the 3% hydrogen peroxide is achieved within 6 hours by a catalytic platinum disk which is located in the contact lens storage case according to the manufacturer's instructions. Miltefosine was dissolved in 5% Ethanol (2 mM stock solution) and tested at 80 μM in Acanthamoeba medium (PYG) or in contact lens disinfection medium.

### Example 2

### Acanthamoeba Cyst Production for Susceptibility Tests

A. castellanii, was isolated from a patient suffering from a severe Acanthamoeba keratitis. Isolation was achieved by inoculating corneal epithelium onto non-nutrient agar plates covered with a 48-h-old culture of Escherichia coli. The isolate was cloned by transferring a single cyst onto a fresh plate with a micromanipulator. Axenisation was achieved by harvesting cysts from parallel cultures and incubating them in 3% HCl overnight in order to eliminate coexisting bacteria. The cysts were then transferred to sterile filtrated PYG (proteose peptone-yeast extract-glucose) that was used as axenic medium further on. Synchronized encystment of the amoebae was achieved by long term storage without the addition of fresh medium. The process of encystment including morphological changes of the cyst wall was observed daily by phase-contrast microscopy. After 14 days the cysts of both strains were in their mature stage. The cysts were harvested by centrifugation (500 g/7 minutes), resuspended in sterile 0.9% NaCl and counted in a BurkerTurk haemocytometer. Of each strain two suspensions were prepared, one with 104 and one with 108 cysts/ml, respectively.

### Example 3

### Performance of the Susceptibility Tests for Acanthamoeba cysts

Tests were performed in 15 ml centrifugation tubes. 100 μl of the respective cyst suspension (10³ cells) and 8 ml of the respective contact lens solution were added per tube. All solutions were used according to the manufacturers' instructions. After a soaking time of 8 hours the pellets (after 500 g/7 minutes centrifugation) of the respective tubes were inoculated onto non-nutrient agar plates covered with a layer of Escherichia coli. The plate cultures were sealed and incubated at 30° C. for 14 days. Amoebic growth was observed daily by phase contrast microscopy. All experiments were carried out in triplicate. The control groups were performed with sterile 0.9% NaCl.

TABLE 1
Viable cells of Acanthamoeba detected on non-nutrient agar plates after
incubation with four commercially available contact lens care solutions
comprising H₂O₂ (1, 2) Polyhexamethylene Biguanide (3), sorbitol (4)
and Bactofongus (5), Miltefosine and contact lens care solutions
comprising Miltefosine respectively, after 8 hours (+, viable cells;
−, no viable cells).

| | | Viable Acanthamoeba |
|---|---|---|
| | Viable Acanthamoeba cells | cells after disinfection |
| | after disinfection with | with commercially avail- |
| | commercially available | able disinfectants com- |
| | disinfectants | prising 80 μM Miltefosine |
| 1 | + | − |
| 2 | + | − |
| 3 | + | − |
| 4 | + | − |
| 5 | + | − |
| Milte- | − | |
| fosine | | |
| 9% NaCl | + | − |

1. A composition for the care of contact lens comprising
an effective amount of miltefosine as an antimicrobial agent for disinfecting one or more contact lenses.

2. The composition according to claim 1, wherein the effective amount of miltefosine is in an amount of between about 5 and about 1500 μM.

3. The composition according to claim 1, wherein the effective amount of miltefosine is in an amount of between about 20 and about 1200 μM.

4. The composition according to claim 1 further comprising a buffer component.

5. The composition according to claim 1 wherein the pH of the solution is in a range of between about 6 and about 8.

6. The composition according to claim 1 further comprising a surfactant.

7. The composition according to claim 1 further comprising a viscosity increasing component.

8. The composition according to claim 1 further comprising a tonicity component.

9. The composition according to claim 1 further comprising a chelating component.

10. The composition according to claim 1 further comprising a protease.

11. The composition according to claim 1 further comprising one or more additional disinfectants.

12. The composition according to claim 1, wherein the composition is a tablet.

13. A method of treating contact lenses comprising the step of:
contacting one or more contact lenses with an effective amount of an miltefosine antimicrobial composition for disinfecting the one or more contact lenses wherein the treating comprises cleaning, chemical disinfecting, storing, rinsing contact lenses or a combination thereof.

14. The method of claim 13 wherein the miltefosine antimicrobial agent affects protozoan organisms, Acanthamoeba or a combination thereof.

15. (canceled)

16. The method of claim 13 wherein the the one or more contact lenses are selected from hard, soft, rigid gas permeable lenses, or soft gas permeable lenses.

17. The method of claim 13, wherein the contact lens is stored in the miltefosine antimicrobial composition for at least 1 minute.

18. A Multi-purpose solution for contact lenses comprising:
an effective amount of miltefosine as an antimicrobial agent for disinfecting one or more contact lenses.

19. A disinfecting Kit for one or more contact lenses containing comprising:
a composition an effective amount of miltefosine as an antimicrobial agent for disinfecting one or more contact lenses,
a rinsing solutions and
a drop delivery tools, preferably pipettes.

20. The composition according to claim 1 further comprising a phosphate buffer component.

21. The composition according to claim 1 further comprising one or more selected from the group selected from cellulose derivatives, polyols, polyethylene oxide containing polymers, polyvinyl alcohol, polyvinyl pyrrolidone, sodium chloride, potassium chloride and calcium chloride, propylene glycol, glycerol, ethylene diamine tetraacetic acid, alkali metal salts of ethylenediaminetetraacetic acid, mixtures and derivatives thereof.

22. The Multi-purpose solution of claim 18 further comprising a buffer component, a surfactant, a viscosity increasing component, a tonicity component, a chelating component, a protease, one or more additional disinfectants or a combination thereof.

23. The kit of claim 21 further comprising a buffer component, a surfactant, a viscosity increasing component, a tonicity component, a chelating component, a protease, one or more additional disinfectants or a combination thereof.

24. The kit of claim 21, wherein the one or more drop delivery tools comprise one or more pipettes.
